(19) United States
(12) Patent Application Publication
(10) Pub. No.: US 2003/0082580 A1
(43) Pub. Date: May 1, 2003

(54) Plant cell culture and selection system

(75) Inventors: Dean Engler, Morago, CA (US); Steven Scofield, West Lafayette, IN (US); Neal Gutterson, Oakland, CA (US); Peter John Balint-Kurti, Walnut Creek, CA (US)

Correspondence Address: FENNEMORE CRAIG, Suite 2600, 3003 N. Central Avenue, Phoenix, AZ, 85012, US

(73) Assignee: DNA Plant Technology Corporation, Oakland, CA

(21) Appl. No.: 10/172,586

(22) Filed: Jun. 13, 2002

Related U.S. Application Data
(60) Provisional application No. 60/303,440, filed on Jul. 6, 2001.
(60) Provisional application No. 60/303,442, filed on Jul. 6, 2001.

(51) Int. Cl. C12Q 1/68; A01H 1/00; C12N 15/82; C12N 5/04

(52) U.S. Cl. 435/6; 435/419; 800/284

(57) ABSTRACT

The present invention provides methods of selecting and transforming plant cells in large scale in vitro liquid cultures. In some methods of the invention, cells are selected that comprise a suppressive nucleic acid sequence that suppresses the effect of a target gene that impairs cellular function in the cell. In other embodiments, the methods are directed to identifying nucleic acids that encode polypeptides that physically interact with one another.

### CROSS-REFERENCES TO RELATED APPLICATIONS

### Background of the Invention

As genomics research in humans and other organisms has advanced, the need to understand the function of the encoded proteins has become of increasing importance. A first level of understanding of protein function is typically an understanding of the function of the protein in isolation. It is well known, however, that biological processes rely on multiprotein complexes. In addition, proteins may inhibit or enhance the activity of other proteins, either directly or indirectly. Thus, identifying proteins which interact, either directly or indirectly, with a given protein is critical to obtaining a better understanding of a protein and how it functions in the cell.

Since many physiologically important interactions are transient or unstable, it is often difficult to obtain information about protein-protein interactions outside the cellular environment. A number of cellular assays have been developed to study direct protein-protein interactions in vivo.

A commonly used assay is the yeast two-hybrid system (see, e.g., Fields, Nature 340:245-6 (1989) and Finley, R. L. JR & Brent R. (1996) in DNA Cloning—Expression Systems: A Practical Approach, eds. Glover D. & Hames B. D (Oxford University Press, Oxford, England), pp. 169-203). This assay requires expressing test protein as a fusion to a DNA-binding domain of a transcriptional activator, and expressing another protein of interest as a fusion to a transcriptional activation domain. If the test proteins interact, a transcriptional activator is reconstituted, resulting in the induction of a reporter gene.

Other assays include the ubiquitin-based split protein sensor assay (U.S. Pat. Nos. 5,585,245 and 5,503,977). This assay is based on cleavage of ubiquitin into two fragments, which are each fused to a different test protein. The re-assembly of the ubiquitin fragments is dependent upon the ability of the two test proteins to interact. Re-assembly of the proper tertiary structure of ubiquitin is detected by the ability of a ubiquitinase to cleave the protein.

Two International applications (WO 98/34120 and WO 00/07038) describe general strategies for designing protein fragment complementation assays (PCAs). These applications specifically describe the use of fusions of test proteins to fragments of dihydrofolate reductase (DHFR). The re-assembly of the protein is detected by the ability of the cell to grow on a selective medium comprising methotrexate.

There is a need in the art to develop useful assays to test protein-protein interactions in plant cells. These efforts have been hindered by the lack of efficient systems for transforming and selecting large numbers of plant cells in vitro. Since, in many cases, it would be desirable to regenerate plants from the selected cells, the system should ideally be suited for this purpose. In addition, it would be useful to detect, not only direct protein-protein interactions, but also detect the ability of a test compound (e.g., a protein) to inhibit or enhance (either directly or indirectly) the activity of a target protein. The present invention addresses these and other needs.

### BRIEF SUMMARY OF THE INVENTION

The present invention provides methods of introducing nucleic acids into plant cells and selecting desired cells in large scale in vitro liquid cultures. In some methods of the invention, cells are selected that comprise a suppressive nucleic acid sequence that suppresses the effect of a target gene that modifies cellular function in the cell. In these methods the methods involve (a) providing a plurality of plant cells in a first liquid culture medium, wherein each such plant cell comprises a promoter operably linked to the target gene; (b) providing a library of nucleic acid molecules, each nucleic acid molecule comprising a promoter operably linked to a test nucleic acid sequence suspected of being a suppressive nucleic acid sequence; (c) introducing the library of test nucleic acids into the plant cells in the liquid culture medium; and (d) selecting plant cells in which cellular function is not modified, thereby selecting plant cells that comprise the suppressive nucleic acid sequence.

Alternatively, the methods of the invention can involve creating a population of transformed plant cells in liquid culture in which endogenous genes are ectopically expressed. The ectopically expressed endogenous genes are tested for their ability to encode proteins that suppress the effect of the target gene that modifies cellular function in the cell. These methods are carried out by selecting plant cells ectopically expressing a suppressive gene that suppresses the effect of a target gene that modifies a cellular function, the method comprising: (a) providing a plurality of plant cells in a first liquid culture medium, wherein each such plant cell comprises a promoter operably linked to the target gene; (b) introducing into the plant cells in the liquid culture recombinant activation tagging constructs comprising an enhancer element, thereby causing ectopic expression of genes in the plant cells; and (c) selecting plant cells in which cellular function is not modified, thereby selecting plant cells in which a suppressive nucleic acid sequence is ectopically expressed.

Alternatively, the methods of the invention can involve modification of cellular function through treatment with a chemical agent. Treatment with a chemical agent can be followed by selection of cells that comprise a suppressive nucleic acid sequence, through transformation with a gene library, that suppresses the effect of the chemical treatment that modifies cellular function in the cell. One embodiment of the method utilizing a chemical agent comprises: (a) providing a plurality of plant cells in a first liquid culture medium, which contains a chemical agent that modifies cellular function; (b) providing a library of nucleic acid molecules, each nucleic acid molecule comprising a promoter operably linked to a test nucleic acid sequence suspected of being a suppressive nucleic acid sequence; (c) introducing the library of test nucleic acids into the plant cells in the liquid culture medium; and (d) selecting plant cells in which cellular function is not modified, thereby selecting plant cells that comprise the suppressive nucleic acid sequence. Another embodiment of the method utilizing a chemical agent comprises: (a) providing a plurality of plant cells in a first liquid culture medium, which includes a chemical agent that modifies cellular function; (b) introducing into the plant cells in the liquid culture recombinant activation tagging constructs comprising an enhancer element, thereby causing ectopic expression of genes in the plant cells; and (c) selecting plant cells in which cellular function is not modified, thereby selecting plant cells in which a suppressive nucleic acid sequence is ectopically expressed.

The particular target gene used in the methods is not critical to the invention. In some embodiments, the target gene impairs cellular function, for example a gene that induces apoptosis in plant cells. Suitable genes, include R genes such as pto^{y207D}. In some embodiments, the target gene is operably linked to an inducible promoter. The particular inducible promoter used is not critical. Exemplary promoters include promoters comprising a bacterial antibiotic resistance operon such as the tet operon. Other suitable promoters include a dexamethasone inducible promoter, a copper inducible promoter, and an ethanol inducible promoter. The test nucleic acid sequences are typically derived from a library of nucleic acids clones that can be either cDNA or genomic DNA.

The particular plant cells are not critical and can be, for example, Nicotiana tabacum. Any standard method of introducing the nucleic acids into the plant cells can be used. Typically, the nucleic acids are introduced into the plant cells using Agrobacterium. In preferred embodiments, the plant cells are in a cycling bioreactor which provides for the production of sufficient independently transformed plants (e.g., millions) such that genome or cDNA libraries can be reasonably screened using a suitable selection.

The invention also provides plant cells comprising a first expression cassette comprising a promoter operably linked to the target gene that that impairs cellular function and a second expression cassette comprising a promoter operably linked to a suppressive nucleic acid sequence that suppresses the effect of the target gene.

The invention also provides method of identifying nucleic acids that encode polypeptides that physically or directly interact with one another. These methods comprise (a) providing a first nucleic acid construct encoding a first fusion protein comprising a first polypeptide fused to a first non-functional fragment of a reporter molecule; (b) providing a second nucleic acid construct encoding a second fusion protein comprising a second polypeptide fused to a second non-functional fragment of a reporter molecule; (c) introducing the first and second nucleic acid constructs into a plant cell in a liquid culture medium; and (d) identifying cells in which reporter molecule activity is present resulting from the physical interaction of the first and second polypeptides.

The nature of the reporter molecule is not critical. It may, for example, confer resistance to a cytotoxic compound such as methotrexate. Alternatively, the reporter molecule may produce a detectable signal, for example, a fluorescent signal.

In some embodiments, the first or the second nucleic acid construct comprises an inducible promoter operably linked to a nucleic acid sequence encoding the first or second fusion protein. The second nucleic acid construct typically comprises nucleic acids from a library of cloned DNA, either cDNA or genomic DNA.

In some embodiments, the reporter molecule is cytotoxic, whereby interaction of the first and second polypeptides results in inhibition of cell growth. In these embodiments, the methods may further comprise the step of adding a test molecule to the plant cells and cells whose growth is not inhibited by the reporter molecule are selected.

### Definitions

Units, prefixes, and symbols can be denoted in their SI accepted form. Numeric ranges are inclusive of the numbers defining the range. The headings provided herein are not limitations of the various aspects or embodiments of the invention which can be had by reference to the specification as a whole. Accordingly, the terms defined immediately below are more fully defined by reference to the specification in its entirety.

As used herein, the term “plant” includes reference to whole plants, plant organs (e.g., leaves, stems, roots, etc.), seeds and plant cells and progeny of same. The class of plants which can be used in the methods of the invention is generally as broad as the class of higher plants amenable to transformation techniques, including both monocotyledonous and dicotyledonous plants.

As used herein “operably linked” includes reference to a functional linkage between a promoter and a second sequence, wherein the promoter sequence initiates and mediates transcription of the DNA sequence corresponding to the RNA sequence which is typically transcribed into a polypeptide. Generally, operably linked means that the nucleic acid sequences being linked are contiguous and, where necessary to join two protein coding regions, contiguous and in the same reading frame.

As used herein, an “expression cassette” is a nucleic acid construct, generated recombinantly or synthetically, with a series of specified nucleic acid elements which permit transcription of a particular nucleic acid in a target cell. The expression cassette can be incorporated into a plasmid, chromosome, mitochondrial DNA, plastid DNA, virus, or nucleic acid fragment. Typically, the expression cassette portion of the expression vector includes, among other sequences, a nucleic acid to be transcribed and a promoter.

As used herein, “a gene that impairs cellular function” refers to a polynucleotide that encodes a polypeptide that is cytotoxic to an extent that kills cells or inhibits cell division or differentiation. Thus, the term “impairs cellular function” includes induction of apoptosis or programmed cell death or other disruption of a cell through perturbation of some function of the cell or by degradation of a component of the cell. By way of example, but not limitation, typical cellular functions in the context of the instant invention are protein synthesis, RNA synthesis, maintenance of osmotic competence, lipid synthesis, DNA synthesis. Typical cellular components subject to degradation in the context of the instant invention are proteins, carbohydrates, membranes, deoxyribonucleic acids, ribonucleic acids.

A “suppressive nucleic acid sequence” is a nucleic acid whose presence in a cell suppresses the effect of a target gene in the cell. The suppression can be mediated in a variety of ways and the specific method of suppression is not critical to the invention. For example, a protein encoded by the suppressive nucleic acid can directly or indirectly inhibit the activity of the protein encoded by the target gene. Alternatively, the suppression may result from homology-dependent suppression of the target gene. Post-transcriptional gene silencing resulting, for example, from the introduction of double-stranded RNA has been described (see, e.g., English et al., Plant Cell 8:179-188 (1996); Waterhouse et al., Proc. Nat'l Acad. Sci. USA 95:13959-13964 (1998)). Antisense technology can also be conveniently used (see, e.g., Heiser et al. Plant Sci. (Shannon) 127:61-69 (1997)). Another method of suppression is sense suppression (see, e.g., Napoli et al., The Plant Cell 2:279-289 (1990); and U.S. Pat. Nos. 5,034,323, 5,231,020, and 5,283,184). Other known methods of suppression include oligonucleotide-based triple-helix formation (see, e.g., Chan and Glazer J. Mol. Medicine (Berlin) 75:267-282 (1997)) and catalytic RNA molecules or ribozymes (Sun et al. Mol. Biotechnology 7:241-251 (1997).

As used herein, “beneficial” includes reference to polynucleotide(s) or polypeptide(s) that impart a protective effect to a cell. In plants, a beneficial effect includes resistance to biotic and abiotic stresses including, but not limited to, plant pathogens, pests, drought, heavy metals, salt, and agricultural chemicals (e.g., herbicides).

An “ectopically expressed” endogenous gene is an endogenous gene that is expressed at levels and under conditions in which it is not normally expressed. For example, the gene can be expressed in different tissues or at different stages in development, than it is normally expressed. As explained below, ectopic expression is typically induced by introducing activation tagging constructs into a plant cell.

As used herein, “heterologous” is a nucleic acid that originates from a foreign species, or, if from the same species, is substantially modified from its original form. For example, a promoter operably linked to a heterologous structural gene is from a species different from that from which the structural gene was derived, or, if from the same species, one or both are substantially modified from their original form. Thus, a “heterologous expression cassette” is one that comprises at least one element not endogenous to the species or sub-species in which it is introduced.

As used herein, “polynucleotide” and “nucleic acid” includes reference to both double stranded and single stranded DNA or RNA. The terms also refer to synthetically or recombinantly derived sequences essentially free of non-nucleic acid contamination. A polynucleotide can be a gene subsequence or a full length gene (cDNA or genomic). Unless specifically limited, the term encompasses nucleic acids containing known analogues of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol Cell. Probes 8:91-98 (1994)). The term nucleic acid is used interchangeably with gene, cDNA, and mRNA encoded by a gene.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(see, e.g., Creighton, Proteins (1984)).

The term “enhanced stability” refers to a polypeptide that has been modified so that one or more of the amino acids has been changed relative to the wild type polypeptide. Such modifications provide enhanced stability to the polypeptide, either alone or in combination with another polypeptide. Enhanced stability includes, e.g., enhanced thermal stability, enhanced activity at lower concentrations, enhanced active site activity, and the like.

As used herein, “functional” includes reference to an activity sufficient to produce a desired effect. Thus, for example, a “functional polypeptide” will have the activity to achieve a desired result, such as lethality in a cell, or the ability of a cell to grow on a selective medium. A “non-functional fragment” of such a polypeptide will lack such activity. A nonfunctional fragment may be, for example, a monomer from a multimeric protein or a subsequence from a monomeric protein.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that initiate transcription only in certain tissues, such as leaves, roots, fruit, seeds, tapetal tissue, anthers, stigmas, or flowers. Such promoters are referred to as “tissue specific”. A “cell type” specific promoter is primarily drives expression in certain cell types in one or more organs, for example, vascular cells in roots or leaves. An “inducible” promoter is a promoter which is under environmental control. Examples of environmental conditions that may effect transcription by inducible promoters include chemical reagents, anaerobic conditions or the presence of light. Tissue specific, cell type specific, and inducible promoters constitute the class of “non-constitutive” promoters. A “constitutive” promoter is a promoter which is active under most environmental conditions.

### DETAILED DESCRIPTION OF THE INVENTION

The present invention is based on the development of new methods that allow for transforming large numbers of plant cells in liquid culture and subsequent selection of cells comprising desired nucleic acids. Briefly, the methods of the invention comprise introducing into plant cells in a liquid medium nucleic acid molecules that are suspected of providing a desired effect. Since the plant cells are in a liquid medium, the selection of desired clones is greatly facilitated. In addition, explants are preferably used to regenerate whole plants from the selected cells. The desired molecules can be selected according to a variety of approaches. In one approach, the ability of the nucleic acids to suppress the effect of a target gene is determined. In another approach, the ability of the nucleic acids to suppress the effect of a chemical agent is determined. For example, in one embodiment, the desired molecule can encode a protein that inhibits the effect of the protein encoded by the target gene or the effect of the chemical agent. In a typical embodiment, the target protein will be a protein that impairs cellular function such as a protein that induces apoptosis. In another typical embodiment, the chemical agent impairs cellular function by inducing apoptosis. In yet another approach, the methods are used to detect direct physical interaction between the proteins encoded by the library and a bait protein.

The invention also provides activation tagging methods for creating a population of transformed plant cells in liquid culture. Briefly, these methods comprise introducing into plant cells in a liquid medium nucleic acid elements capable of inserting into the plant genome (e.g., a T-DNA vector or transposable element). The nucleic acid elements comprise enhancer elements that can cause ectopic expression of endogenous gene. The ectopic expression of endogenous genes allows for the selection of cells expressing desired endogenous genes.

Recombinant Vectors

The invention is practiced using known methods, which are well described in the scientific and patent literature. The various genes and nucleic acids useful in the invention, whether RNA, cDNA, genomic DNA, or hybrids thereof, may be isolated from a variety of sources and genetically engineered, expressed recombinantly, or may be synthesized in vitro according to standard techniques. Techniques for manipulation of nucleic acids, such as site specific mutagenesis, generating libraries, subcloning into expression vectors, labeling probes, sequencing DNA, DNA hybridization are described in the scientific and patent literature, see e.g., Sambrook, ed., Molecular Cloning: a Laboratory Manual (2nd ed.), Vols. 1-3, Cold Spring Harbor Laboratory, (1989); Current Protocols in Molecular Biology, Ausubel, ed. John Wiley & Sons, Inc., New York (1997) (“Ausubel”); and, Laboratory Techniques in Biochemistry and Molecular Biology: Hybridization With Nucleic Acid Probes, Part I. Theory and Nucleic Acid Preparation, Tijssen, ed. Elsevier, N.Y. (1993).

Typically, the recombinant expression vectors of the invention will comprise an expression cassette, which comprises a promoter operably linked to a coding sequence of interest. As noted above, recombinant expression cassettes comprising test sequences need not encode a protein. In these embodiments, the coding sequence will be designed to provide homology-dependent suppression of the target gene (e.g., by linking the coding sequence to the promoter in an antisense orientation). If proper polypeptide expression is desired, those of skill will recognize that additional sequences may be included in the expression cassette. For example, a polyadenylation region at the 3′-end of the coding region can be included. (See, Li Plant Physiol.115:321-325 (1997), for a review of the polyadenylation of RNA in plants). The polyadenylation region can be derived from the natural gene, from a variety of other plant genes, or from T-DNA.

The expression vectors may also contain sequences permitting replication of the cassette in vivo, e.g., plants, eukaryotes, or prokaryotes, or a combination thereof, (e.g., shuttle vectors) and selection markers for the selected expression system, e.g., plant, prokaryotic or eukaryotic systems. For example, the marker may encode antibiotic resistance, particularly resistance to chloramphenicol, kanamycin, G418, bleomycin, hygromycin, or herbicide resistance, such as resistance to chlorosulfuron or Basta, to permit selection of those cells transformed with the desired DNA sequences, see for example, Blondelet-Rouault Gene 190:315-317 (1997); Aubrecht J. Pharmacol. Exp. Ther. 281:992-997 (1997).

In the methods of the invention, typically a library of nucleic acids are tested for a desired effect. The preparation of expression vectors for these sequences is routine. A constitutive promoter will typically be used to drive expression of the test nucleic acids. Such promoters are active under most environmental conditions and states of development or cell differentiation. Examples of constitutive promoters include the cauliflower mosaic virus (CaMV) 35S transcription initiation region, the 1′- or 2′-promoter derived from the T-DNA of Agrobacterium tumafaciens, various ubiquitin or polyubiquitin promoters derived from, inter alia, Arabidopsis (Sun and Callis, Plant J., 11(5):1017-1027 (1997)), the mas, Mac or DoubleMac promoters (described in U.S. Pat. No. 5,106,739 and by Comai et al., Plant Mol. Biol. 15:373-381 (1990)) and other transcription initiation regions from various plant genes known to those of skill in the art. Such genes include for example, ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), and Gpc2 from maize (GenBank No. U45855, Manjunath et al., Plant Mol. Biol. 33:97-112 (1997)).

The library of test nucleic acids can be derived from any number of sources. Typically, the nucleic acids will be cDNA or genomic clones. Preparation of cDNA and genomic libraries are well known in the art. In the case of cDNAs, different tissues may be used as source material depending upon the method used. For example, different tissues, or the same tissue under different conditions such as drought or disease conditions, may be more or less likely to express a desired suppressive gene in the case of embodiments in which suppression of a target gene is desired. Similarly, the bait protein may determine source of cDNAs used in methods of detecting direct interaction between proteins. Thus, the cells used to prepare the test nucleic acids need not be of plant origin and can be derived from any organism such as prokaryotes (e.g., bacteria), fungi, yeast, insects, or mammals.

A specialized library can also be used. For example, and particularly from plants whose genome or transcriptome has been well defined, a library that contains a defined subset of known genes can be used. For example, the library may contain all known transcription factors, all known kinase genes, all known phosphatase genes, and the like. Such a library can be assembled from cDNA clones or genomic clones of each known sequence.

In addition, further variation in the library can be achieved using known techniques. A variety of diversity generating protocols are available and described in the art. The procedures can be used separately, and/or in combination to produce variants of the library of nucleic acids, as well variants of encoded proteins. A variety of diversity generating procedures for generating modified nucleic acid sequences are known. These include error-prone PCR (see, e.g., Leung et al., Technique 1, 11-15 (1989)), DNA shuffling (see, e.g., Minshull and Stemmer Current Opinion in Chemical Biology 3:284-290 (1999); Stemmer In: The Encyclopedia of Molecular Biology. VCH Publishers, New York. pp.447-457 (1996), and U.S. Pat. Nos. 5,605,793, 5,811,238, 5,830,721, 5,834,252, and 5,837,458) stEP (see, e.g., Zhao et al. Nat. Biotechnol. 16:258-261 (1998)) and ITCHY (Ostermeier et al. Nature Biotechnol. 17: 1205-1209 (1999)).

Other mutational methods of generating diversity include, for example, site-directed mutagenesis (Ling et al. Anal Biochem. 254(2): 157-178 (1997) and Smith Ann. Rev. Genet. 19:423-462 (1985) point mismatch repair (Kramer et al. Cell 38:879-887 (1984)), mutagenesis using repair-deficient host strains (Carter et al. Nucl. Acids Res. 13: 4431-4443 (1985).

As noted above, in one aspect of the invention, the methods of the invention can be used in two general approaches. The two approaches of the invention differ in the manner in which the test nucleic acids are tested. In one set of embodiments, the test nucleic acids are tested for their ability to suppress the effects of a target gene that modifies cellular function in the cell. In other embodiments, the test nucleic acids are ligated to sequences encoding non-functional fragments of a reporter molecule and tested for their ability to encode a protein that physically interacts with a “bait” protein fused to another non-functional fragment of the reporter. Expression cassettes appropriate for each of these embodiments are discussed below.

In another aspect of the invention, the population of cells expressing genes to be tested for the ability to suppress the effect of a target gene that modifies cellular function is generated through the introduction of a DNA element (e.g., a T-DNA or transposable element) comprising transcriptional enhancer sequences at one or both ends. Using these methods, endogenous genes of the plant cell are ectopically expressed (i.e. at levels and under conditions in which they are not normally expressed). Enhancer sequences are conveniently derived from strong constitutive promoters such as the CaMV 35S promoter, the Figwort Mosaic Virus 34S promoter, the mannopine synthase promoter, and the like. Such enhancer elements can be incorporated into T-DNA and used in activation tagging procedures as described in Walden et al. Plant Mol Biol 26:1521-1528 (1994) and Weigel et al. Plant Physiol. 4:1003-1013 (2000)).

Methods for Identifying Suppressive Polynucleotides

In methods of identifying suppressive nucleic acids, the target gene can be operably linked to an inducible promoter. The inducible promoter is used to control expression of the target gene so that the modifications of cellular function can be controlled. Thus, the affects of the target gene product are induced at the time, or some time following the time that, the library of test nucleic acids is introduced into the cells. This is particularly useful if the target gene impairs cellular function.

A number of inducible promoters are known. The promoters can be induced in response to any environmental stimulus, such as chemical reagents, light, temperature, oxygen level, and the like. Typically, the promoters are induced in response to chemical reagents. These reagents include, e.g., herbicides, synthetic auxins, or antibiotics which can be applied the plant cells. For a review of suitable inducible promoters, see Gatz, Annu. Rev. Plant Physiol. Plant Mol. Bio. 48:89-108 (1997). Examples of inducible promoters include promoters comprising bacterial antibiotic resistance operons, such as the tet operon (see, e.g., Gatz et al. The Plant Journal 2:397-404 (1992)). These promoters are induced in response to tetracycline. Other examples include ethanol-inducible promoters (Caddick et al. Nature Biotechnol. 16:177-180 (1998)), copper-inducible promoters (Mett et al. Proc. Natl. Acad. Sci. U.S.A. 90:4567-4571 (1993)), and glucocorticoid-inducible (e.g., dexamethasone) promoters (Aoyoma et al. The Plant Journal 11:605-612 (1997)). The maize In2_{—}2 promoter, which is activated by benzenesulfonamide herbicide safeners (De Veylder et al. Plant Cell Physiol. 38:568-577 (1997)) can also be used. Synthetic promoters that are induced by dexamethasone and repressed by tetracycline have also been developed (Bohner and Gatz Mol Gen Genet 264:860-870 (2001)).

The target gene used in the methods will depend upon the particular response being investigated. The only requirement is that the target gene modify cellular function in such a way to produce an easily identifiable phenotype. For example, the target gene can encode a protein involved in the biosynthesis or response to plant hormones, e.g., ethylene, cytokinins, auxins, gibberellins, and the like. For example, it is known that ethylene inhibits callus formation and regeneration of plant cells. The inability to form callus or regenerate whole plants can therefore be a basis for selection. Similarly, other hormones are known to alter cell division and/or regeneration of plants in culture. Examples of suitable genes/selection systems include ETR (ethylene receptor) and auxin/cytokinin perception genes. A specific gene system is the IAAH gene in conjunction with indoleacetamide media supplementation. In other embodiments, genes that encode proteins involved in pigment formation (e.g., anthocyanin synthesis) can be used. Plant cells are then selected based on the ability to produce the pigment. Examples of suitable pigment biosythesis genes include chalcone synthase, dihydrofolate reductase and the Lc gene from corn.

In other embodiments, the target gene impairs cellular function. Typically, such a gene will encode a protein that is cytotoxic and cells are selected for their ability to survive. For example, proteins and genes that control programmed cell death can be investigated using the methods of the invention. There are two general modes of cell death. Death that requires de novo gene expression is termed “programmed cell death” (PCD). Alternatively, in “necrosis” the cell is a passive victim of various forms of trauma causing loss of membrane integrity. Studies of morphologies of dying animal cells have demonstrated that several modes of programmed cell death exist: 1) apoptosis, which is characterized by loss of structure of the nucleus, fragmentation of chromosomal DNA and activation of caspases; 2) cytoplasmic degenerative PCD, in which the nucleus may or may not degenerate, but the cytoplasm is consumed by autophagic organelles; and 3) lysosomal degenerative PCD, in which the cytoplasm is lost but without the involvement of an organelle or other cell (Jones, Trends in Plant Science 5: 225-230 (2000)). Much less evidence about the existence of different classes of PCD exists in plant systems. However, PCD involved in the formation of tracheary elements is argued to be nonapoptotic. Groover et al. Protoplasma 196: 197-211 (1997)). Many insect, nematode, and mammalian genes involved in the pathway of apoptosis have been cloned and characterized.

In these embodiments, a nucleic acid that encodes a protein that induces cell death is used as the target gene. Suitable genes include, for example, plant disease resistance genes associated with a hypersensitive cell death response (R genes). Evidence suggests that the localized cell death associated with the hypersensitive response (HR) triggered by plant disease resistance genes (R-genes) involves the process of apoptosis. These genes have the advantage that the hypersensitive cell death response is induced by pathogen derived elicitor polypeptides encoded by avirulence genes. Thus, control of expression of the avirulence genes can be used to induce lethality. Examples of polypeptides include avirulence/resistance gene combinations include the AVR elicitor polypeptides from Cladosporium fulvum and the corresponding resistance genes, Cf from Lycopersicon (e.g., Cf2/Avr2, Cf4/Avr4, Cf5/Avr5, and Cf9/Avr9, see, Jones et al. Science 266:789-793 (1994) and Hammond-Kosack and Jones Plant Cell 8:1773-1791 (1996)). In preferred embodiments, the AVR peptide is linked to a sequence targeting it to the apoplast (see, e.g., Hammond-Kosack et al. Proc. Natl. Acad. Sci. USA 91:10445-10449). Other avirulence/resistance gene combinations include the tomato Pto gene and the Pseudomonas syringae avrPto avirulence gene (Martin et al. Science 262:1432 (1993), the RPS2 gene of Arabidopsis thaliana confers resistance to P. syringae that express the avrRpt2 avirulence gene (Bent et al. Science 265:1856-1860 (1994)), and the tobacco N gene and TMV replicase (Padgett et al. Molecular Plant Microbe Interactions 10709-715 (1997)). Alternatively, in the cases of the Pto (Rathjen et al., EMBO J18:3232-40 (1999)), and Mi1 (Hwang et al. Plant Cell. 12:1319-29 (2000)) mutant derivatives have been identified that are constitutively active. An example is the pto^{Y207D} described by Rathjen et al., above. In these embodiments cell death may be triggered by directly controlling expression of the activated R gene, with out the need for co-expression of the elicitor gene.

In addition, non-plant genes can be used as target genes. Examples include genes and their encoded proteins that are associated with apoptotic pathways in mammalian cells. Many of these genes that control apoptosis are highly conserved. Some mammalian gene products can stimulate cell death, whereas others can prevent cell death. For example, Bcl-2 can inhibit apoptosis whereas Bax, a member of the Bcl-2 family, can promote apoptosis. Identification of the genes that regulate cell death has propelled the study of apoptosis to biomedical research. Recently, a number of studies have implicated that AP-1 (activating protein-1) and MAP (mitogen activated protein) kinases are involved in apoptotic cell death. Other pro-apoptotic animal genes of interest include EGL-1 (Conradt et al. Cell. 93:519-29 (1998), Caspase-9 (Li et al. Cell. 91:479-89 (1997)), CED-3 (Yuan et al. Cell. 75:641-52 (1993)), and APAF-1 (Zou et al. Cell. 90:405-13 (1997)).

Although many genes have been identified whose products are essential for activating or inhibiting apoptosis, there may be more, as yet unidentified, cell death-related proteins. Proteins involved in these pathways can be identified in the methods of the present invention.

As noted above, the methods of the invention can also be used to identify nucleic acids that suppress the effect of chemical agents. The chemical agent can be any agent that modifies cellular function. Plant hormones can be used, in which case suppressive nucleic acids would be those that suppress the effect of the plant hormone. A range of plant hormone classes are well known in the art, including auxins, cytokinins, gibberellins, abscissic acid, ethylene, jasmonic acid, brassinosteroids. Other chemical agents involved in plant physiological responses can also be used, including salicylic acid. Chemical agents that perturb cellular metabolism can also be used, including agents that may perturb programmed cell death pathways (such as camptothecin or paraquat). Chemical agents that when applied in high concentrations provide stresses to plant cells can also be used, including salts (e.g., NaCl), sugars (e.g., glucose, sucrose, mannitol).

Methods for Detecting Physical Interactions Between Proteins

In these embodiments, both the test nucleic acids (the “prey” nucleic acids) and the target gene (the “bait”) are prepared as fusions to a non-functional fragment of a reporter molecule. The ability of the two gene products to physically interact is detected by the re-assembly of a functional reporter molecule. Each expression cassette (i.e. the expression cassette encoding the bait and that encoding the prey) is individually functional but the product of each cassette alone does not provide the desired effect. It takes the re-assembly of the translated polypeptides from the individual expression cassettes to result in the detectable phenotype. Thus, each fragment is individually non-functional.

The reporter molecules of the invention can be any molecule that provides a detectable phenotype. A detectable phenotype can be, for example, colorimetric, fluorimetric, or ability to grow on a selective medium. In some embodiments, the reporter, itself impairs cellular function and the methods further include a step of adding test compounds that are tested for their ability to inhibit the reporter. Typically, the reporter will be a protein that confers resistance to a cytotoxic compound, such as an antibiotic, herbicide and the like. An exemplary reporter, dihydrofolate reductase (DHFR) and selection of media containing methotrexate, is described below. Other suitable reporter molecules include, barnase, barstar, adenosine deaminase, thymidine kinase, luciferase, green flourescent protein, glucoronidase, and the like. The ubiquitin based split protein sensor assay can also be used in the present invention (see, U.S. Pat. Nos. 5,585,245 and 5,503,977). See also Wilmink et al. Plant Mol. Biol. Rep. 11: 165-185 (1993); Bowen, Markers for plant gene transfer. In Kung, S. D., Wu, R. (eds) Transgenic Plants, Vol.1. pp89-146. Academic Press, New York (1993); Joersbo et al., Pl. Cell Rep. 16: 219-221 (1996); and PCT 98/34120.

The reporter molecules of the present invention typically consist of polypeptides derived from overlapping or non-overlapping subsequences of a single functional protein which provides the desired phenotype when re-assembled in the cell. Alternatively, the polypeptides can be separate functional proteins from distinct loci. Thus, for example the reporter can be can be either a monomeric protein or a multimer derived from distinct loci. In the case of multimeric proteins, the non-functional fragments are typically monomers of the protein. The reporter molecule is usually an enzyme that catalyzes a reaction that provides the detectable phenotype (e.g., a fluorescent product or that detoxifies a toxic compound). Typically, the two peptides are from non-overlapping or minimally overlapping (e.g., 50, 35, 20, 15, 10, 5 or less) subsequences from a single protein.

The non-functional fragments may also be modified according to standard methodology to produce polypeptides with desired properties, e.g., enhanced thermal stability, enhanced subunit association, enhanced activity at lower concentrations and the like. These peptides can also be modified to produce conservatively modified variants. The modification of the polypeptides can be achieved, by techniques known to those skilled in the art such as random or site-specific mutagenesis of the nucleic acids that encode the polypeptides. Using such methods of mutagenesis, genetically modified peptides are then assayed for reconstitution of activity in vivo. Activity at lower concentrations or at higher temperatures is measured by comparing the genetically modified and the original peptides.

The secondary and tertiary structure of a host of proteins and the processes of protein folding are known to those of skill and provide the basis for designing appropriate reporter molecules. The design of non-functional polynucleotides or their encoded polypeptides can be achieved by a number approaches well known to the skilled artisan. In the instant invention, these polynucleotides or polypeptides, when co-expressed in a cell, will confer the detectable phenotype. As noted above, the amino acid sequence of the fragments may be modified for a variety of purposes. The amino acid changes can be determined by examination of the original protein and the known amino acid interactions based on the protein structure as revealed through a range of physical techniques. In addition, the amino acid changes can be determined by random mutagenesis and screening of a combinatorial library of protein products. Alternatively, the amino acid changes can be determined by completely random mutagenesis and selection, using chemical treatments, PCR-induced mutagenesis, or other similar mutagenic treatments known to those skilled in the art.

The partial coding sequences derived from the original protein coding sequences is selected to retain activity of the reconstituted protein as well as a suitable level of stability with respect to environmental perturbations such as temperature changes. Several general routes can be taken to determining effective partial coding sequences.

A number of proteins have been separated into distinct, resolvable domains through partial proteolysis. This is a rapid way to determine suitable coding sequences for the two non-functional polynucleotides or polypeptides of the instant invention. Proteases initially cleave an intact protein at exposed residues, often ones that are part of exposed loops not involved in specific domains. Following partial proteolysis and analysis of the resulting peptide fragments by polyacrylamide gel electrophoresis to confirm that a simple digestion resulted, residual activity is evaluated. If activity is retained, the peptides are separated to determine whether neither peptide retains activity separately, and subsequently whether activity can be reconstituted upon remilling. Sequencing of the amino and carboxy termini of the two (or more) fragments reveals how to engineer the partial coding sequences in the instant invention.

In situations where a number of sequences are available for proteins with the same function it is possible to identify regions that are not well conserved in all proteins. In combination with predictive analysis of secondary structure, it is possible to identify regions of the protein that are good candidates for separation into separate peptides. Such regions retain unaltered principal secondary structural features, such as alpha helices and beta-sheets. Within such regions, a number of possible replacement and coding sequence variants can be tested using an assay either for protein function in vitro, or for function, or for in vivo lethality.

When a three-dimensional structure is available, from x-ray crystallography, NMR spectroscopy, etc., a more precise determination of candidate regions that would comprise the partial peptides of a single functional reporter molecule is possible. Analysis of the interactions between individual amino acids in a three-dimensional structure reveals sub-domains of the original protein that have the potential to be separated and yet to bind to each other, and which sub-domains are likely to be non-functional when present separately. Additionally, analysis of these interactions reveals which amino acids, located between suitable sub-domains, are not involved in specific interactions with other amino acids in a way that would permit replacement with a methionine residue. Such an analysis is aided by additional sequence data for proteins with a high proportion of sequence relatedness to the starting protein. This provides additional evidence concerning residues that can be replaced with a methionine residue.

As noted above, in some embodiments, the reporter molecules impair cellular function. Exemplary polypeptides useful in these embodiments are ribonucleases such as barnase (Mauguen et al., Nature 297:162-64 (1982)), binase (Pavlovsky et al., FEBS Lett. 162:167-70 (1983)), Ribonuclease T1 (Fujii et al., Biosci. Biotechnol. Biochem. 59:1869-1874 (1995)), nucleases such as colicin E9 (Wallis et al., Eur. J. Biochem 220:447-54 (1994)) or BamHI (Newman et al., Science 269:656-63 (1995)), and proteases such as subtilisin BPN′ (Eder et al., J. Mol. Biol. 233:293-304) or other members of the subtilisin family. Other polypeptides for creating cell toxicity or inhibition include those which produce toxic substances, disrupt cell function, suppress genes required by the cell (such as by using anti-sense, sense suppression, or ribozymes), and disruption of mitochondrial function. In particularly preferred embodiments, the polypeptide is derived from a separate subsequence of a ribonuclease such as barnase. For barnase, the minimal length of each polypeptide is at least 20 amino acids. Generally, the extent of overlap of barnase polypeptides will be no more than 5 amino acids (see, e.g., WO 98/32325).

Introduction of Vectors into Plant Cells

Once the constructs are prepared they are introduced into plant cells in a liquid medium, preferably using a liquid bioreactor as described here. The plant cells can be either explants (i.e. derived directly from whole plants) or tissue culture cells that are typically maintained in culture.

The present invention has use over a broad range of types of plants, including species from the genera Cucurbita, Rosa, Vitis, Juglans, Fragaria, Lotus, Medicago, Onobrych is, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum and Datura.

The DNA constructs of the invention may be introduced into the genome of the desired plant host by a variety of conventional techniques. For example, the DNA construct may be introduced directly into the genomic DNA of the plant cell using techniques such as electroporation of plant cell protoplasts, or the DNA constructs can be introduced directly to plant tissue using ballistic methods, such as DNA particle bombardment.

The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is co-cultured with the bacteria. Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983).

Transformed plant cells which are derived by any of the above transformation techniques can be cultured to produce undifferentiated cells (e.g., callus) or regenerate a whole plant which possesses the transforming DNA. Such techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Callus production and regeneration can also be obtained from plant callus, explants, organs, or parts thereof. Such regeneration techniques are described generally in Klee et al. Ann. Rev. of Plant Phys. 38:467-486 (1987).

All Liquid Culture System

As noted above, a preferred method of introducing the test nucleic acids into the plant cells is in a high throughput, bioreactor method using an Agrobacterium tumefaciens vector. This provides a rapid and reliable method for transforming large numbers of plant cells with a library of test nucleic aids. Typically, the introduction of the constructs and all subsequent manipulations of the cells are done in liquid culture. Use of an all liquid system allows addition or deletion of media components (e.g., plant hormones, salts, sugars, antibiotics, selective agents, and the like) or replacement of spent media with fresh media without the need to transfer the cultures from one solidified medium to another. In preferred methods, a cycling bioreactor in which the media reservoir is separated from the culture vessel is used. In the cycling bioreactor, plant tissue culture media is periodically pumped from a reservoir bottle into a bioreactor bottle containing the plant cells which are typically left explants. The media is then allowed to immediately drain back into the reservoir bottle leaving the cells coated with fresh media but not submerged. This process is repeated several times (preferably 6) each day.

### DESCRIPTION OF THE SPECIFIC EMBODIMENTS

### Example 1

Assembly of Constructs and Transgenic Plants Providing Regulated Cell-death

In this example the constitutively active form of Pto, pto^{Y207D} (Rathjen et al., supra) was utilized as a genetic trigger of death. To construct a regulated pto^{Y207D} gene, an XbaI-NcoI restriction fragment encoding pto^{Y207D} was ligated into the expression cassette constructs pNG5612 and pNG6013. In these cassettes gene expression was driven by the Smas promoter (see, Ni et al. Plant J. 7: 661-676 (1995)) which has been engineered to be repressible by the tetracycline repressor protein that regulates the tet operon of E. coli. These promoters were constructed so that immediately downstream of the transcriptional initiation site were multiple copies of the operator sequence from the tet operon of E. coli and were given the name STOP. pNG5612 contains two copies of the operator sequence (2×STOP), while pNG6013 has 3 copies (3×STOP). The resulting constructs, pBN002 and pBN003 were then digested with PvuII which releases the 2×STOP: pto^{Y207D}: OCS3′ and 3×STOP: pto^{Y207D}: OCS3′ fragments. The PvuII fragments were cloned into the XbaI site of pUC119 which was made blunt ended by treatment with Klenow enzyme and dNTPs. Clones were selected in which the STOP promoter was proximal to the SalI site of the pUC119 multiple cloning site, and were designated pBN011 and pBN012 which carry 2× and 3×STOP: pto^{Y207D}: OCS3′, respectively. The 2× and 3×STOP: pto^{Y207D}: OCS3′ fragments were excised from pBN011 and pBN012 with SmaI and SalI digestion and ligated into SmaI-SalI digested pWTT2161, a binary T-DNA vector that carries a pNOS:NPTII marker for selection in planta. The resulting binary plasmids, pBN013 and pBN0014 were transferred to Agrobacterium tumefaciens strain LBA4404.

The plasmids pBN013 and pBN014 are transformed into a line of Nicotiana tabacum cv. Petite Havana transformed with the spectinomycin selected T-DNA construct pBN009, which line expresses the tetracycline repressor gene from the Smas promoter. This line expresses sufficient levels of tet repressor to provide efficient repression of the 2×STOP:β-glucuronidase (GUS) reporter construct, and when tetracycline is added to the media, de-repression of the reporter gene occurs.

Transgenic plants carrying pBN013 and pBN014 are selected using kanamycin. Leaf disks are punched from leaves of the transgenics and placed in tetracycline containing media to test for tetracycline inducible cell-death. From results of this assay one line carrying pBN013+pBN009 and one harboring pBN014 +pBN014 are chosen for testing in the high-throughput functional assay for plant cDNAs able to suppress pto^{Y207D} dependent death.

Assembly of T-DNA Library Expressing Arabidopsis cDNAs

To obtain a cDNA with very complete representation of the Arabidopsis transcriptome, RNA was prepared from a variety of tissues and treatments. Total RNA was isolated from Arabidopsis leaves, flowers and callus tissues, and also from Arabidopsis plants infected with Botrytis, Pseudomonas syringae and Phytophthora capsici. PolyA+ RNA was then purified. Double stranded cDNA is generated using the Clontech SMART cDNA kit. The cDNAs are digested from the plasmid with SfiI and directionally cloned into SfiI digested pBK88, a binary T-DNA that expresses the cDNAs from the CaMV35S promoter, and provides selection in planta using chlorsulfurone. The ligation products are electroporated directly into Agrobacterium tumefaciens strain LBA4404. The electroporation mixture is plated on LB Agar containing 1 μg/ml tetracycline.

High-Throughput Transformation of T-DNA cDNA Library

Leaf explants of the doubly transformed Petite Havana plants described above are prepared by harvesting sterile leaves, placing them in a blender containing MS media, and blending for about 5 seconds on high. Approximately 25,000 of these leaf explants are then mixed with a mixed culture of Agrobacterium tumefaciens containing the above-described library of Arabidopsis genes which has been grown to a density of 6×10⁸ cells per milliliter. This mixture of Agrobacterium and leaf explants is then dispensed into 10 sterile 10 liter vessels (approximately 2500 explants per vessel) and co-cultured for 3 days at 26° C. Following the co-culture period, about 2 liters of TCl medium supplemented with 500 mg/l carbenicillin to eradicate Agrobacterium is pumped into each vessel and immediately allowed to drain back out leaving the explants coated with media but not submerged (TCl medium is composed of MS salts with 20 g/l sucrose, 100 mg/l inostol and 3 mM N morpholino ethanesulfonic acid and the phytohormones Benzyladenine and indolacetic acid added at 0.5 mg/l and 2 mg/l respectively; the pH is adjusted to 5.8.) The explants are periodically immersed into the same media by repeating the pumping and draining process 6 times per day for the next 3 days, followed by replacement of the media with fresh TCl media and resuming the cycling process for an additional 3 days.

Induction of Cell-Death

At this point expression of the pto^{Y207D} gene is derepressed by replacing the TCl media with TCl media containing 1 mg/L Tetracycline, and the cycling process describe above is resumed. Calli and shoots which form and/or survive and grow in the presence of the Tetracycline are harvested and may be transferred to solidified media for further growth. It is likely that some of these plant materials contain genes derived from the Arabidopsis T-DNA library which confer resistance to cell death triggered by the expression of the pto^{Y207D} gene.

Recovery of cDNAs from Surviving Calli

It should be confirmed that the pto^{Y207D} gene is induced in all surviving calli. Therefore, subclones of all surviving calli are grown again on tetracycline containing media, then RNA is prepared and tested on northern blots to confirm whether they display tetracycline dependant expression of pto^{Y207D.} The Arabidopsis cDNAs are then isolated from all clones that pass this test. This is accomplished by preparing genomic DNA from the calli tissue and performing PCR utilizing primers that anneal to the cDNA expression cassette at positions flanking the cDNA insert.

### Example 2

Using the High-throughput Transformation System to Detect Protein-protein Interactions in Planta

Interaction cloning is a useful technology for determining the identities of gene products that physically interact with one another and potentially participate in the same biological pathway. The yeast two-hybrid system (Y2H) is a well known system for interaction cloning. In the Y2H system, selected bait proteins are expressed in yeast as fusions to the DNA binding domain of transcription factors, i.e, yeast GAL4, or other proteins that bind defined DNA sequences with high specificity, i.e, E. coli LexA. Genes encoding other products thought to interact with the “Bait’ or libraries of cDNAs are then expressed as fusions to the transcriptional activation domain of a transcription factor. If the “bait” and “prey” gene products physically interact with one another, they bring together the DNA binding and transcriptional activation domains attached to the bait and prey fusions and reconstitute a functional transcription factor that is then able to activate expression of a marker gene, whose function can be selected for screened in yeast. This assay is well suited for screens of large numbers cDNA prey fusions, but suffers from several limitations. For interactions to be detected, the bait and prey fusion proteins must be successfully transported to the yeast nucleus. In addition, if the interaction requires other proteins to stabilize or to modify the interacting proteins, suitable forms of the auxiliary proteins must be encoded by the yeast genome.

Assembly of Constructs and Transgenic Plants Expressing “Bait Protein” Fusions

The leucine zipper domain of GCN4 has been shown to dimerize in vivo, and can therefore, be used to demonstrate the efficacy of this in planta protein-protein interaction detection system.

Assembly of T-DNA Expressing GCN4 Leucine Zipper:C′ Terminal DHFR “Prey Protein”

The GCN4 leucine zipper domain (A₂₃₂-R₂₈₁) was PCR amplified from Saccharomyces cerevisiae so that it contains an NcoI site adding a methionine residue to the N-terminus of the resulting protein, and such that the linker amino acid sequence (GGGS)₂ is added to the C-terminus. After confirming the sequence of the PCR product, overlap-extension PCR is performed to join the leucine zipper domain to the N-terminal fragment of murine dihydrofolate reductase (DHFR). After further sequence confirmation, this overlap-extension product is digested with NcoI and XbaI and directionally cloned into the binary vector pCT4001. The resulting binary T-DNA, pCT4002, expresses the GCN4 leucine zipper domain from the CaMV35S promoter so that it is fused to the N′ terminal fragment of DHFR and can be selected in planta using kanamycin. The same leucine zipper fragment is also utilized in an overlap-extension PCR reaction with a DNA fragment encoding the C-terminal portion of DHFR. After confirming the sequence of this PCR product, the sequence is digested with Ncol and XbaI and cloned into the chlorsulfuron selected binary pCT4003, creating the plasmid pCT4004. The plasmid pCT4004 expresses a protein product consisting of the GCN4 leucine zipper domain fused at its C-terminus to the C-terminal fragment of DHFR from the CaMV35S promoter. pCT4002 and pCT4004 are transferred to Agrobacterium tumefaciens strain LBA4004 by electroporation. The electroporation mixtures are plated on LB Agar containing 1 μg/ml tetracycline.

High-Throughput Transformation of Tobacco with a Mixture of “Prey” and Control T-DNAs

Transgenic Petite Havana plants are generated that express the pCT4002 TDNA. To demonstrate that T-DNAs encoding interacting protein pairs can be selected in planta, a mixed culture of Agrobacterium tumefaciens is created with a mixture of one part Agrobacterium containing pCT4004 to 10,000 parts Agrobacterium carrying the control plasmid pCT4003. Explants from the pCT4002 expressing line are prepared by harvesting sterile leaves, placing them in a blender containing MS media, and blending for about 5 seconds on high. Approximately 5,000 of these leaf explants are then incubated with the 1:10,000 mixed culture of Agrobacterium, described above, which is grown to a density of 6×10⁸ cells per milliliter. The Agrobacterium and leaf explants are then dispensed into 2 sterile 10 liter vessels (approximately 2,500 explants per vessel) and co-cultured for 3 days at 26° C. Following the co-culture period, about 2 liters of plant tissue culture media (TC formula 1) is pumped into each vessel and immediately allowed to drain back out leaving the explants coated with media but not submerged. The explants are periodically immersed into the same media by repeating the pumping and draining process 6 times per day for the next 3 days, followed by replacement of the media with fresh TCl media and resuming the cycling process for an additional 3 days.

Methotrexate Selection of Transformants Expressing pCT4002 and pCT4004

After three days, the composition of the TCl media for one of the 10 liter vessels is changed so that it now contains 20-1000 μg/L methotrexate. The media of the other vessel is changed to contain 200 μg/ml kanamycin.

Demonstrating That Surviving Calli Harbor Insertions of pCT4002 and pCT4003

RNA's are prepared from 20 methotrexate resistant calli and from 20 kanamycin resistant calli. The RNA's are run out on duplicate agarose-formaldehyde gels and transferred to northern blot filters. The duplicate filters are probed with the N-terminal and C-terminal fragments of DHFR. When probed with the N-terminal and C-terminal DHFR probes, all RNA's from methotrexate resistant calli are shown to express the full-length mRNA's expected of pCT4002 and pCT4003. However, RNA's isolated from the kanamycin selected calli show only the full-length message encoded by pCT4002. The fact that none of the kanamycin selected calli display the pCT4004 mRNA indicates that reconstitution of DHFR activity occurs only in plant cells that express both the GCN4 leucine zipper-N-terminal DHFR and the GCN4 leucine zipper-C-terminal DHFR fusions.

### Split Negative Selection

If a particular protein-protein interaction detected by such a screen is confirmed to occur in vivo during the normal function of a biochemical pathway of interest, the existence of a high-throughput assay for the interaction can be a powerful tool for identifying molecules that disrupt the interaction. To utilize the assay in this way, however, protein-protein interaction must activate a lethal selectable marker, so that only plant material in which the interaction fails to occur will survive. A preferred method to create such a lethal selection for protein-protein interaction is to utilize the two-component Barnase system that is described in WO 98/32325.

### Example 3

Using the High-throughput Transformation System to Identify in Planta cDNAs Encoding Proteins That Physically Interact with One Another

The product of the ADAGIO1 gene (ADO1) is a key component of circadian regulation in Arabidopsis. The yeast two-hybrid system has been used to show that ADO1 physically interacts with the two photoreceptor proteins, phyB and CRY1 (Jarillo et. al., Nature 410: 487-490 (2001). The invention disclosed herein is used to demonstrate that the same protein-protein interactions can be identified via an in planta screen of an Arabidopsis cDNA library.

The ADO1 coding sequence is PCR amplified from Arabidopsis cDNA so that it contains an NcoI restriction site at the initiation codon, and such that the linker amino acid sequence (GGGS)₂ is added to the C-terminus. After confirming the sequence of the ADO1 PCR product, overlap-extension PCR is performed to join ADO1 to the N-terminal fragment of murine dihydrofolate reductase (DHFR). After further sequence confirmation, this overlap-extension product is digested with Ncol and XbaI and directionally cloned into the kanamycin selected binary vector pCT4001. The resulting plasmid, pCT4005, expresses ADO1 from the CaMV35S promoter so that it is fused in frame, at its C-terminus, to the N-terminal fragment of DHFR.

A cDNA library with very complete representation of the Arabidopsis transcriptome RNA was prepared from a variety of tissues. Total RNA was isolated from Arabidopsis leaves, flowers and callus tissues. PolyA+ RNA was then purified. Double stranded cDNA is generated using the Clontech SMART cDNA kit. The cDNAs are digested with SfiI and directionally cloned into SfiI digested pCT4006, a chlorsulfuron selected binary T-DNA. pCT4006 is designed so that cDNAs can be directionally cloned downstream of the C-terminal fragment of DHFR, which is under the control of the CaMV35S promoter. Therefore, the inserted cDNAs are expressed as N-terminal fusions to the C-terminal fragment of DHFR. The ligation products are electroporated directly into Agrobacterium tumefaciens strain LBA4404. The electroporation mixture is plated on LB Agar containing 1 μg/ml tetracycline.

Transgenic Petite Havana plants are created that express the pCT4005 ADO1:N-terminal DHFR fragment fusion.. Explants from a pCT4006 expressing line are prepared by harvesting sterile leaves, placing them in a blender containing MS media, and blending for about 5 seconds on high. Approximately 25,000 of these leaf explants are then incubated with a mixed culture of Agrobacterium tumefaciens containing the above-described library of Arabidopsis genes which culture is grown to a density of 6×10⁸ cells per milliliter. This mixture of Agrobacterium and leaf explants is then dispensed into 10 sterile 10 liter vessels (approximately 2500 explants per vessel) and co-cultured for 3 days at 26° C. Following the co-culture period, about 2 liters of plant tissue culture media (TC formula 1) is pumped into each vessel and immediately allowed to drain back out leaving the explants coated with media but not submerged. The explants are periodically immersed into the same media by repeating the pumping and draining process 6 times per day for the next 3 days, followed by replacement of the media with fresh TCl media and resuming the cycling process for an additional 3 days.

Methotrexate Selection of Transformants Expressing pCT4002 and pCT4004

After three days, the composition of the TCl media for the 10 liter vessels is changed so that it contains 20-1000 μg/L methotrexate. Methotrexate resistant calli and shoots are propagated. After sufficient growth, genomic DNA is prepared from the resistant calli and shoots and PCR amplification is performed to recover the cDNA sequences.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of selecting plant cells comprising a suppressive nucleic acid sequence that suppresses the effect of a target gene that modifies a cellular function, the method comprising:
(a) providing a plurality of plant cells in a first liquid culture medium, wherein each such plant cell comprises a promoter operably linked to the target gene;
(b) providing a library of nucleic acid molecules, each nucleic acid molecule comprising a promoter operably linked to a test nucleic acid sequence suspected of being a suppressive nucleic acid sequence;
(c) introducing the library of test nucleic acids into the plant cells in the liquid culture medium; and
(d) selecting plant cells in which cellular function is not modified, thereby selecting plant cells that comprise the suppressive nucleic acid sequence.

2. The method of claim 1, wherein the target gene impairs cellular function.

3. The method of claim 2, wherein the target gene induces programmed cell death in plant cells.

4. The method of claim 3, wherein the target gene is an R gene.

5. The method of claim 4, wherein the R gene is pto^{y2O7D}.

6. The method of claim 1, wherein the promoter operably linked to the target gene is an inducible promoter and the method further comprises the step of inducing expression of the target gene.

7. The method of claim 6, wherein the inducible promoter comprises a bacterial antibiotic resistance operon.

8. The method of claim 7, wherein the antibiotic resistance operon is a tet operon and the step of inducing expression of the target gene is carried out by contacting the cells with tetracycline.

9. The method of claim 6, wherein the inducible promoter is a dexamethasone inducible promoter.

10. The method of claim 6, wherein the inducible promoter is a copper inducible promoter.

11. The method of claim 6, wherein the inducible promoter is an ethanol inducible promoter.

12. The method of claim 1, wherein the test nucleic acid sequences are cDNAs.

13. The method of claim 1, wherein the test nucleic acid sequences are genomic DNA.

14. The method of claim 14, wherein the plant cells are Nicotiana tabacum.

15. The method of claim 1, wherein the library of nucleic acid molecules is introduced into the plant cells using Agrobacterium.

16. The method of claim 1, wherein the plant cells are in a cycling bioreactor which provides for adding a second liquid culture medium without removing the plant cells from the first liquid culture medium.

17. The method of claim 16, wherein the second culture medium is different from the first culture medium.

18. A plant cell comprising a first expression cassette comprising a promoter operably linked to a target gene that modifies cellular function and a second expression cassette comprising a promoter operably linked to a suppressive nucleic acid sequence that suppresses the effect of the target gene.

19. The plant cell of claim 18, wherein the target gene impairs cellular function.

20. The plant cell of claim 18, wherein the target gene induces programmed cell death in plant cells.

21. The plant cell of claim 20, wherein the target gene is an R gene.

22. The plant cell of claim 21, wherein the R gene is pto^{y207D}.

23. The plant cell of claim 18, wherein the promoter operably linked to the target gene is an inducible promoter.

24. The plant cell of claim 23, wherein the inducible promoter comprises a bacterial antibiotic resistance operon.

25. The plant cell of claim 25, wherein the antibiotic resistance operon is a tet operon.

26. The plant cell of claim 23, wherein the inducible promoter is a dexamethasone inducible promoter.

27. The plant cell of claim 24, wherein the inducible promoter is a copper inducible promoter.

28. The plant cell of claim 23, wherein the inducible promoter is an ethanol inducible promoter.

29. The plant cell of claim 18, which is Nicotiana tabacum.

30. A method of identifying nucleic acids that encode polypeptides that physically interact with one another, the method comprising:
(a) providing a first nucleic acid construct encoding a first fusion protein comprising a first polypeptide fused to a first non-functional fragment of a reporter molecule;
(b) providing a second nucleic acid construct encoding a second fusion protein comprising a second polypeptide fused to a second non-functional fragment of a reporter molecule;
(c) introducing the first and second nucleic acid constructs into a plant cell in a liquid culture medium; and
(d) identifying cells in which reporter molecule activity is present resulting from the physical interaction of the first and second polypeptides.

31. The method of claim 30, wherein the reporter molecule confers resistance to a cytotoxic compound.

32. The method of claim 31, wherein the reporter molecule is dihydrofolate reductase.

33. The method of claim 32, where in the cytotoxic compound is methotrexate.

34. The method of claim 30, wherein the reporter molecule produces a detectable signal.

35. The method of claim 34, wherein the reporter molecule produces a fluorescent signal.

36. The method of claim 30, wherein the reporter molecule is monomeric and the first and second non-functional fragments are each a polypeptide derived from the reporter molecule.

37. The method of claim 30, wherein the first or the second nucleic acid construct comprises an inducible promoter operably linked to a nucleic acid sequence encoding the first or second fusion protein.

38. The method of claim 37, wherein the inducible promoter comprises a bacterial antibiotic resistance operon.

39. The method of claim 38, wherein the antibiotic resistance operon is a tet operon.

40. The method of claim 37, wherein the inducible promoter is a dexamethasone inducible promoter.

41. The method of claim 37, wherein the inducible promoter is a copper inducible promoter.

42. The method of claim 37, wherein the inducible promoter is an ethanol inducible promoter.

43. The method of claim 30, wherein the second nucleic acid construct comprises cDNA.

44. The method of claim 30, wherein the second nucleic acid construct comprises genomic DNA.

45. The method of claim 30, wherein the plant cells are Nicotiana tabacum.

46. The method of claim 30, wherein the first and second nucleic acid constructs are introduced into the plant cells using Agrobacterium tumefaciens.

47. The method of claim 30, wherein the plant cells are in a cycling bioreactor which provides for adding a second liquid culture medium without removing the plant cells from the first liquid culture medium.

48. The method of claim 47, wherein the second culture medium is different from the first culture medium.

49. The method of claim 30, wherein the reporter molecule is cytotoxic, whereby interaction of the first and second polypeptides results in inhibition of cell growth.

50. The method of claim 49, further comprising the step of adding a test molecule to the plant cells and step (d) is carried out by selecting cells whose growth is not inhibited by the reporter molecule.

51. A method of selecting plant cells ectopically expressing a suppressive gene that suppresses the effect of a target gene that modifies a cellular function, the method comprising:
(a) providing a plurality of plant cells in a first liquid culture medium, wherein each such plant cell comprises a promoter operably linked to the target gene;
(b) introducing into the plant cells in the liquid culture recombinant activation tagging constructs comprising an enhancer element, thereby causing ectopic expression of endogenous genes in the plant cells; and
(c) selecting plant cells in which cellular function is not modified, thereby selecting plant cells in which a suppressive nucleic acid sequence is ectopically expressed.

52. The method of claim 51, wherein the target gene impairs cellular function.

53. The method of claim 52, wherein the target gene is an R gene.

54. The method of claim 53, wherein the R gene is pto^{y207D}.

55. The method of claim 51, wherein the promoter operably linked to the target gene is an inducible promoter and the method includes the step of inducing expression of the target gene.

56. The method of claim 51, wherein the plant cells are Nicotiana tabacum.

57. The method of claim 51, wherein the recombinant activation constructs are transposons.

58. The method of claim 51, wherein the recombinant activation constructs are T-DNAs.

59. The method of claim 51, wherein the recombinant activation constructs are introduced into the plant cells using Agrobacterium.

60. The method of claim 51, wherein the plant cells are in a cycling bioreactor which provides for adding a second liquid culture medium without removing the plant cells from the first liquid culture medium.

61. The method of claim 60, wherein the second culture medium is different from the first culture medium.

62. A method of selecting plant cells comprising a suppressive nucleic acid sequence that suppresses the effect of a target gene that modifies a cellular function, the method comprising:
(a) providing a plurality of plant cells in a first liquid culture medium, which contains a chemical agent that modifies cellular function;
(b) providing a library of nucleic acid molecules, each nucleic acid molecule comprising a promoter operably linked to a test nucleic acid sequence suspected of being a suppressive nucleic acid sequence;
(c) introducing the library of test nucleic acids into the plant cells in the liquid culture medium; and
(d) selecting plant cells in which cellular function is not modified, thereby selecting plant cells that comprise the suppressive nucleic acid sequence.

63. A method of selecting plant cells comprising a suppressive nucleic acid sequence that suppresses the effect of a target gene that modifies a cellular function, the method comprising:
(a) providing a plurality of plant cells in a first liquid culture medium, which contains a chemical agent that modifies cellular function;
(b) introducing into the plant cells in the liquid culture recombinant activation tagging constructs comprising an enhancer element, thereby causing ectopic expression of genes in the plant cells; and
(c) selecting plant cells in which cellular function is not modified, thereby selecting plant cells in which a suppressive nucleic acid sequence is ectopically expressed.
